SEQUENCE LISTING (2 sequences)

SEQ ID NO: 1 - DNA, 20 bp, synthetic construct
  source 1..20: mol_type = other DNA; note = Primer 27F
agagtttgatcctggctcag

SEQ ID NO: 2 - DNA, 19 bp, synthetic construct
  source 1..19: mol_type = other DNA; note = Primer 1492R
ggttaccttgttacgactt

(19) United States
(12) Patent Application Publication
(10) Pub. No.: US 2026/0103676 A1
(43) Pub. Date: Apr. 16, 2026

(54) Deodorizing Strain, Deodorizing Microbial Agent, and Use Thereof

(71) Applicant: China Agricultural University, Beijing (CN)

(72) Inventors: Ruixue Chang, Beijing (CN); Yuxin Sun, Beijing (CN); Chenjunnan Zhou, Beijing (CN); Yanming Li, Beijing (CN); Guoxue Li, Beijing (CN); Yizhan Qiu, Beijing (CN); Yang Yang, Beijing (CN); Lingxiao Wang, Beijing (CN); Anqi Wang, Beijing (CN); Runze Chen, Beijing (CN)

(21) Appl. No.: 19/355,851

(22) Filed: Oct. 10, 2025

(30) Foreign Application Priority Data
Oct. 12, 2024 (CN) 202411423358.5

(51) Int. Cl. C12N 1/20; A61L 9/012; C05G 3/90

(52) U.S. Cl. CPC C12N 1/205; A61L 9/012; C05G 3/90

(57) ABSTRACT

The present invention relates to a deodorizing strain, a deodorizing microbial agent, and use of the deodorizing strain and the deodorizing microbial agent. The deodorizing strain is selected from Providencia vermicola CM1, Pseudochrobactrum asaccharolyticum CM5, and Providencia sp. CM18 and exhibits a prominent deodorizing effect. The deodorizing microbial agent including the three deodorizing strains can improve a sulfur removal rate to nearly 80% while maintaining a high ammonia removal rate, which solves the problem that there are low sulfur removal rates in previous studies. Moreover, the deodorizing microbial agent can be easily applied with a low cost, which effectively addresses the issue that the traditional deodorizing microbial agent exhibits inadequate efficacy and slow response when used in the traditional stacking and composting processes for livestock and poultry manure and household waste.

### CROSS-REFERENCE TO RELATED APPLICATION

This patent application claims priority to and the benefit of the Chinese Patent Application No. 202411423358.5 filed with the China National Intellectual Property Administration on Oct. 12, 2024, the disclosure of which is incorporated by reference herein in its entirety as part of the present application.

### REFERENCE TO SEQUENCE LISTING

A computer readable XML file entitled “GWP20250701678_sequence listing”, which was created on Aug. 22, 2025, with a file size of about 3,053 bytes, contains the sequence listing for this application, has been filed with this application, and is hereby incorporated by reference in its entirety.

### TECHNICAL FIELD

The present disclosure belongs to the technical field of microbial agents and environmental engineering using the microbial agents, and specifically relates to a deodorizing strain, a deodorizing microbial agent, and the use of the deodorizing strain and the deodorizing microbial agent.

### BACKGROUND

While providing high-quality meat, eggs, and dairy products, a substantial amount of livestock and poultry manure is produced in the livestock and poultry breeding industry. The malodor issue caused by livestock and poultry manure is increasingly prominent. Animal excreta, animal dander, moldy bedding, feed, dust-laden drainage ditches, wastewater treatment facilities, and among the others are major sources of odor emissions. Odorous gases from composting or livestock and poultry production primarily include volatile sulfur compounds, ammonia, and volatile amines, volatile fatty acids, phenols, and indoles. These compounds are characterized by intense odors and low odor thresholds, posing certain risks to both the surrounding environment and human health. NH₃ and H₂S are the main components in odors from livestock and poultry breeding. NH₃ is generated primarily due to the bacterial decomposition of livestock and poultry excreta and bedding materials and to the action of enteric bacteria in animals. H₂S is produced primarily due to the degradation of sulfur-containing organic matters in fresh livestock and poultry manure by anaerobic microorganisms. H₂S is colorless and highly volatile, and has a rotten egg smell. Therefore, controlling the odor pollution caused by livestock and poultry manure has become a critically urgent issue.

The treatment methods for malodorous gases from livestock and poultry manure include physical, chemical, and microbial treatment methods. However, the physical and chemical treatment methods are costly, technically demanding, and prone to causing secondary pollution. In contrast, due to advantages such as low-cost deodorization, simple operations, and superior effect, the microbial treatment method has become the predominant method for deodorizing livestock and poultry manure. In microbial deodorization technologies, malodorous substances are degraded based mainly on the physiological metabolic activities of various beneficial microorganisms to produce carbon dioxide, water, inorganic salts, etc., thereby reducing odor generation.

There are some functional microorganisms with deodorizing effects in compost, biogas slurry, and biogas residue. These functional microorganisms mainly include Bacillus subtilis, Bacillus lichenformis, Alcaligenes faecalis, and Pseudomonas spp. Currently, a plurality of microbial agents are commonly combined to enhance the odor mitigation at home and abroad. Studies have shown that a composite deodorizing microbial agent exhibits a deodorization effect for both fresh and aged manure and allows significant removal of NH₃, H₂S, and volatile organic acids. However, limited deodorizing strains with single action effects are currently identified and can hardly meet contemporary odor emission standards.

### SUMMARY

An objective of the present disclosure is to provide a deodorizing strain, a deodorizing microbial agent, and the use of the deodorizing strain and the deodorizing microbial agent. The present disclosure identifies three new deodorizing strains. The three deodorizing strains and a composite deodorizing microbial agent produced based on the three deodorizing strains exhibit superior, stable, and long-lasting deodorization effects.

The present disclosure provides a deodorizing strain, including Providencia vermicola CM1, Pseudochrobactrum asaccharolyticum CM5, or Providencia sp. CM18, where the accession numbers of Providencia vermicola CM1, Pseudochrobactrum asaccharolyticum CM5, and Providencia sp. CM18 are CGMCC No. 31631, CGMCC No. 31632, and CGMCC No. 31633, respectively.

The present disclosure also provides a deodorizing microbial agent including a deodorizing strain, where
the deodorizing strain includes one or more strains selected from the group consisting of Providencia vermicola CM1, Pseudochrobactrum asaccharolyticum CM5, and Providencia sp. CM18; and
the accession numbers of Providencia vermicola CM1, Pseudochrobactrum asaccharolyticum CM5, and Providencia sp. CM18 are CGMCC No. 31631, CGMCC No. 31632, and CGMCC No. 31633, respectively.

Preferably, the deodorizing microbial agent includes a microbial suspension of the deodorizing strain and/or a fermentation broth of the deodorizing strain.

Preferably, the deodorizing microbial agent includes a fermentation broth of Providencia vermicola CM1, a fermentation broth of Pseudochrobactrum asaccharolyticum CM5, and a fermentation broth of Providencia sp. CM18; and the fermentation broth of Providencia vermicola CM1, the fermentation broth of Pseudochrobactrum asaccharolyticum CM5 and the fermentation broth of Providencia sp. CM18 are in a volume ratio of (0.5-2.0):(0.5-2.0):(0.5-2.0).

Preferably, viable counts of Providencia vermicola CM1, Pseudochrobactrum asaccharolyticum CM5, and Providencia sp. CM18 in the deodorizing microbial agent are higher than or equal to 10⁸ cfu/mL.

The present disclosure also provides the use of the deodorizing strain described in the above technical solution or the deodorizing microbial agent described in the above technical solution in treating odor.

Preferably, the odor includes one or more components selected from the group consisting of NH₃, H₂S, and a volatile organic acid.

Preferably, the odor includes an odor generated from livestock and poultry manure and/or household waste.

The present disclosure also provides a method for removing an odor, including spraying the deodorizing microbial agent described in the above technical solution on a material carrying the odor, where a spraying amount of the deodorizing microbial agent is 5% to 15% of the mass of the material carrying the odor.

Preferably, the material carrying the odor includes livestock and poultry manure and/or household waste.

### Beneficial Effects

The present disclosure provides a deodorizing strain, including Providencia vermicola CM1, Pseudochrobactrum asaccharolyticum CM5, or Providencia sp. CM18. The accession numbers of Providencia vermicola CM1, Pseudochrobactrum asaccharolyticum CM5, and Providencia sp. CM18 are CGMCC No. 31631, CGMCC No. 31632, and CGMCC No. 31633, respectively. The deodorizing strain of the present disclosure exhibits a prominent deodorizing effect. On this basis, a composite deodorizing microbial agent is produced by mixing the three strains, and in this composite deodorizing microbial agent, there are no antagonistic effects among the three strains. Due to the synergistic interactions among different deodorizing strains, the composite deodorizing microbial agent achieves the optimal average removal efficiency for NH₃ and H₂S and exhibits a stable and long-lasting deodorizing effect. Experimental results reveal that, compared with each of the deodorizing strains alone, the composite deodorizing microbial agent including the three deodorizing strains improves the sulfur removal rate to nearly 80% while maintaining a high ammonia removal rate, which solves the problem that there are low sulfur removal rates in previous studies. Moreover, the deodorizing microbial agent of the present disclosure can be easily applied with a low cost, which effectively addresses the issue that the traditional deodorizing microbial agent exhibits inadequate efficacy and slow response when used in the traditional stacking and composting processes for livestock and poultry manure and household waste. Therefore, the deodorizing microbial agent can effectively mitigate the environmental pollution in the treatment of feces and similar waste.

### Biological Deposit Information

Providencia vermicola CM1: Providencia vermicola CM1 is taxonomically classified under Providencia vermicola, and was deposited in the China General Microbiological Culture Collection Center (CGMCC), Institute of Microbiology, Chinese Academy of Sciences at No. 1 West Beichen Road, Chaoyang District, Beijing, China on Aug. 7, 2024, with an accession number of CGMCC No. 31631.

Pseudochrobactrum asaccharolyticum CM5: Pseudochrobactrum asaccharolyticum CM5 is taxonomically classified under Pseudochrobactrum asaccharolyticum, and was deposited in the China General Microbiological Culture Collection Center (CGMCC), Institute of Microbiology, Chinese Academy of Sciences at No. 1 West Beichen Road, Chaoyang District, Beijing, China on Aug. 7, 2024, with an accession number of CGMCC No. 31632.

Providencia sp. CM18: Providencia sp. CM18 is taxonomically classified under Providencia sp., and was deposited in the China General Microbiological Culture Collection Center (CGMCC), Institute of Microbiology, Chinese Academy of Sciences in No. 1 West Beichen Road, Chaoyang District, Beijing, China on Aug. 7, 2024, with an accession number of CGMCC No. 31633.

### BRIEF DESCRIPTION OF THE DRAWINGS

To explain the technical solutions in the embodiments of the present disclosure or in the prior art clearly, the accompanying drawings required in the embodiments will be briefly described below.

FIG. 1 shows the NH₃ removal rates of different strains within 8 days of secondary screening in Example 1.

FIG. 2 shows the H₂S removal rates of different strains within 8 days of secondary screening in Example 1.

FIG. 3 shows the NH₃ removal rates of a composite deodorizing microbial agent at different spraying amounts in Example 2.

FIG. 4 shows the H₂S removal rates of the composite deodorizing microbial agent at different spraying amounts in Example 2.

FIG. 5 shows the average removal rates of the composite deodorizing microbial agent at different spraying amounts within 8 days in Example 2.

### DETAILED DESCRIPTION OF THE EMBODIMENTS

The present disclosure provides a deodorizing strain, including Providencia vermicola CM1, Pseudochrobactrum asaccharolyticum CM5, or Providencia sp. CM18. The accession numbers of the Providencia vermicola CM1 strain, the Pseudochrobactrum asaccharolyticum CM5 strain, and the Providencia sp. CM18 strain are CGMCC No. 31631, CGMCC No. 31632, and CGMCC No. 31633, respectively.

The deodorizing strains in the present disclosure are isolated from chicken manure compost and pig manure compost samples. Through 16S rRNA sequencing and homology analysis, it has been found that the Providencia vermicola CM1 strain, the Pseudochrobactrum asaccharolyticum CM5 strain, and the Providencia sp. CM18 strain have similarity degrees of 100%, 100%, and 99.929% to Providencia vermicola, Pseudochrobactrum asaccharolyticum, and Providencia sp., respectively, which confirms their taxonomic classification. The Providencia vermicola CM1, the Pseudochrobactrum asaccharolyticum CM5, and the Providencia sp. CM18 are biologically deposited.

Pseudochrobactrum asaccharolyticum and Providencia sp. have been frequently reported in studies related to the remediation of pesticide-contaminated soil, heavy metal-contaminated soil, and organic compound-contaminated soil and the wastewater treatment, but have not been reported in the field of deodorization. In the present disclosure, new functions of Pseudochrobactrum asaccharolyticum and Providencia sp. are discovered, providing a new direction for exploration of deodorizing strains to some extent.

The present disclosure also provides a deodorizing microbial agent. The deodorizing microbial agent includes one or more strains selected from the deodorizing strains described in the above technical solution, preferably two or three strains selected from the group consisting of the deodorizing strains, and more preferably three strains selected from the group consisting of the deodorizing strains. The deodorizing microbial agent of the present disclosure preferably includes a microbial suspension of the deodorizing strain and/or a fermentation broth of the deodorizing strain, and more preferably is the fermentation broth of the deodorizing strain. The deodorizing microbial agent of the present disclosure preferably includes a fermentation broth of the Providencia vermicola CM1 strain, a fermentation broth of the Pseudochrobactrum asaccharolyticum CM5 strain, and a fermentation broth of the Providencia sp. CM18 strain. The fermentation broth of the Providencia vermicola CM1 strain, the fermentation broth of the Pseudochrobactrum asaccharolyticum CM5 strain, and the fermentation broth of the Providencia sp. CM18 strain are in a volume ratio of preferably (0.5-2.0):(0.5-2.0):(0.5-2.0), more preferably 1:1:1. Viable counts of the Providencia vermicola CM1 strain, the Pseudochrobactrum asaccharolyticum CM5 strain, and the Providencia sp. CM18 strain in the deodorizing microbial agent are preferably higher than or equal to 10⁸ cfu/mL. The present disclosure has no specific limitation on preparation methods for the microbial suspension of the deodorizing strain and the fermentation broth of the deodorizing strain, and preparation methods for fermentation broths and microbial suspensions of Pseudochrobactrum asaccharolyticum and Providencia sp. known in the art can be adopted.

The present disclosure also provides the use of the deodorizing strain described in the above technical solution or the deodorizing microbial agent described in the above technical solution in treating odor. In the present disclosure, the odor preferably includes one or more components selected from the group consisting of NH₃, H₂S, and a volatile organic acid, and more preferably includes NH₃ and/or H₂S. In the present disclosure, the odor preferably includes an odor generated from livestock and poultry manure and/or household waste. The odor generated from the household waste preferably includes odor generated during a composting and/or a landfill process of the household waste.

The present disclosure also provides a method for removing an odor, including: the deodorizing microbial agent described in the above technical solution is sprayed on a material carrying the odor for deodorization. A spraying amount of the deodorizing microbial agent is 5% to 15% of the mass of the material carrying the odor. In the present disclosure, the spraying amount of the deodorizing microbial agent is preferably 5% to 10% and more preferably 10% of the mass of the material carrying the odor. In the present disclosure, the material carrying the odor preferably includes livestock and poultry manure and/or household waste. In the present disclosure, the deodorization is conducted preferably for 8 days or less. In the present disclosure, there are no specific limitations on the method, and the specific process of spraying and the conventional methods and specific processes for spraying in the art may be adopted.

In order to further illustrate the present disclosure, the technical solutions provided by the present disclosure are described in detail below in connection with the accompanying drawings and examples, but these examples should not be understood as limiting the claimed scope of the present disclosure.

### Example 1

### 1. Screening of Deodorizing Strains

### 1) Strain Sources

The strains adopted in the present disclosure were derived from chicken manure compost and pig manure compost samples.

### 2) Enrichment and Isolation of Strains

10 g of chicken manure compost sample from the Shangzhuang Experimental Station of China Agricultural University was added to a sterile conical flask with 90 mL of sterile water and glass beads, and enrichment culture was conducted for 30 min with shaking at 30° C. and 180 r·min⁻¹. The enrichment culture solution was diluted by a 10-fold dilution method. 0.1 mL of diluted sample was pipetted and coated evenly on each solid plate (NB medium), allowed to stand for 20 min, and then incubated at a constant temperature of 30° C. for 24 hours. Results were observed.

10 g of pig manure compost sample from the Shangzhuang Experimental Station of China Agricultural University was inoculated into 100 mL of NH₃-selective medium and H₂S-selective medium, respectively, and cultured for 2 days on a shaker at 30° C. and 180 r·min⁻¹ to produce an acclimatization culture solution. 10 mL of acclimatization culture solution was pipetted and inoculated into 100 mL of fresh selective medium for the second-generation acclimation. The enrichment and acclimation were conducted consecutively for 4 generations. An enrichment culture solution of each sample was diluted by a 10-fold dilution method. 0.1 mL of diluted sample was pipetted and coated evenly on each solid plate, and then incubated at a constant temperature of 30° C. for 24 hours. Results were observed.

A total of 46 strains were isolated, with 33 strains isolated from the chicken manure compost sample and 13 strains isolated from the pig manure compost sample.

Formulas of the media adopted were as follows:

NH₃-selective medium: 50.0 g of sucrose, 10.0 mL of ammonia water, 2.0 g of KH₂PO₄, 0.5 g of MgSO₄·7H₂O, 0.1 g of FeSO₄, 5.0 mL of 1% ZnSO₄, 2.0 g of NaCl, and 1,000 mL of distilled water, with a pH unadjusted.

H₂S-selective medium: 5.0 g of glucose, 0.5 g of K₂HPO₄, 1.0 g of KNO₃, 0.5 g of MgCl₂, 0.5 g of NaCl, 0.5 g of NH₄Cl, 1.0 g of Na₂CO₃, 0.01 g of FeCl₂, and 1,000 mL of distilled water, with a pH unadjusted.

Nutrient broth agar medium (NB medium): 10.0 g of peptone, 0.5 g of sodium chloride, 3.0 g of beef extract powder, 15.0 g of agar, and 1,000 mL of distilled water, with a pH of 7.3±0.2.

### 3) Purification of Strains

The plate streaking method was adopted. Single colonies that grew well and had different colors and morphologies were picked with an inoculating loop from each environment on a plate, inoculated on a corresponding plate, and further cultured at the same temperature. Each strain was purified four rounds by the three-zone plate streaking method to produce a pure strain.

### 4) Primary Screening of Deodorizing Strains

100 mL of NH₃-selective medium was taken and added to a shake flask, and ammonia water was added in a percent 25% of the volume of the NH₃-selective medium. An isolated strain was activated, and 500 μL of activated strain solution was inoculated into the shake flask. The shake flask was sealed and subjected to constant-temperature incubation on a shaker at 30° C. and 180 r/min consecutively for 5 days. A change in the strain solution was observed. If the strain solution turned turbid, it indicated that the strain had ability to degrade NH₃. If the strain solution remained transparent, it indicated that the strain could not directly utilize NH₃.

200 mL of H₂S-selective medium was taken and added to a shake flask. A strain was activated, and 500 μL of activated strain solution was inoculated into the shake flask. A 50 mL sterile small beaker was placed in the shake flask, 12 mL of 25% H₂SO₄ was added to the small beaker, and 4 g of FeS was added to the small beaker. The shake flask was then immediately sealed and incubated on a shaker at 30° C. and 200 r/min consecutively for 5 days. The strain solution was observed. If the strain solution turned turbid, it indicated that the strain had an ability to degrade H₂S. If the strain solution remained transparent, it indicated that the strain could not directly utilize H₂S.

Results of the primary screening are shown in Table 1.

TABLE 1
Primary screening results

| | | Number of | | Number of | |
|---|---|---|---|---|---|
| | Total | strains | | strains | |
| | number of | capable of | | capable of | |
| | isolated | removing | Isolation | removing | Isolation |
| — | strains | NH₃ | efficiency/% | H₂S | efficiency/% |
| Result | 46 | 24 | 52.17% | 19 | 41.30% |

Turbidity levels of the NH₃-selective medium and the H₂S-selective medium after the primary screening were graded (mild turbidity, moderate turbidity, and severe turbidity). The mild turbidity is defined as a state in which the medium is slightly pale-yellow or translucent, indicating poor growth performance of a strain. The moderate turbidity is defined as a state in which the medium is pale-yellow, indicating moderate growth performance of a strain. The severe turbidity is defined as a state in which the medium is yellow or off-white and becomes obviously turbid, indicating strong growth performance of a strain. Results are shown in Tables 2 and 3. In terms of the turbidity grading for the NH₃-selective medium, 5 strains led to mild turbidity, 11 strains led to moderate turbidity, and 8 strains led to severe turbidity. In terms of the turbidity grading for the H₂S-selective medium, 3 strains led to mild turbidity, 10 strains led to moderate turbidity, and 6 strains led to severe turbidity. It was found that many strains exhibited a prominent NH₃-removing effect, but few strains exhibited a H₂S-removing effect.

TABLE 2
Turbidity grading for NH₃-selective medium

| Mild turbidity | Moderate turbidity | Severe turbidity |
|---|---|---|
| CM12 | CM4 | CM1 |
| CM22 | CM5 | CM2 |
| CM26 | CM6 | CM3 |
| CM29 | CM8 | CM7 |
| CM35 | CM9 | CM17 |
| — | CM15 | CM18 |
| — | CM21 | CM23 |
| — | CM28 | CM34 |
| — | CM29 | — |
| — | CM30 | — |
| — | CM32 | — |

TABLE 3
Turbidity grading for H₂S-selective medium

| Mild turbidity | Moderate turbidity | Severe turbidity |
|---|---|---|
| CM1 | CM2 | CM4 |
| CM10 | CM3 | CM5 |
| CM36 | CM6 | CM9 |
| — | CM12 | CM23 |
| — | CM16 | CM28 |
| — | CM17 | CM32 |
| — | CM18 | — |
| — | CM21 | — |
| — | CM22 | — |
| — | CM34 | — |

Based on the turbidity levels of the NH₃ and H₂S-selective media in Tables 2 and 3, and the differences in morphology and growth conditions among strains, it can be seen that 13 strains exhibit excellent ability to remove both NH₃ and H₂S.

### 5) Secondary Screening of Deodorizing Strains

The 13 strains primarily selected were used to conduct an 8-day secondary-screening culture experiment with pig manure. NH₃ and H₂S-absorbing solutions were tested every 2 days to determine the deodorizing effect of each strain.

Specific operations were as follows: The different strains were cultured in an NB liquid medium at 30° C. and 180 r·min⁻¹ until a viable count was higher than or equal to 10⁸ cfu/mL. Then, the resulting strain solution was inoculated at a volume ratio of 1000 into a 1 L plastic tank with a lid, including 200 g of pig manure, and stirring was conducted using a glass rod. A small 50 mL beaker containing 20 mL of 2% boric acid-containing absorption solution was placed in each plastic tank for NH₃ absorption, and the plastic tank was sealed with double-layer plastic wrap and then covered with the lid. In a control group (CK), an equal amount of sterile water was adopted. Three replicates were set for each group. A 50 mL small beaker with 20 mL of alkaline zinc-ammonium complex solution was placed in each plastic tank for H₂S absorption. The amount of NH₃ released was determined using a boric acid absorption-titration method, and a removal rate of NH₃ under laboratory culture conditions was calculated. The amount of H₂S released was determined using a zinc-ammonium complex salt colorimetric method, and the removal rate of H₂S was calculated. Three replicates were set for each treatment. The amounts of NH₃ and H₂S released were measured every 2 days, with a measurement period of 8 days.

Determination method of NH₃: An amount of NH₃ released was measured by a boric acid absorption-based Kjeldahl method. 20 mL of 2.0% boric acid solution was used to absorb NH₃ from an experimental group, then two drops of methyl red-bromocresol green indicator were added, and titration was conducted with a sulfuric acid standard solution until a color changed from blue green to light red. The amount of the sulfuric acid standard solution consumed was recorded. A concentration of NH₃ could be calculated according to the formula: C=(c×v×2×1000×17)/V In this formula, C represents a concentration of NH₃, in a unit of mg/L; c represents a molar concentration of the sulfuric acid standard solution, in a unit of mol/L; v represents a volume of the sulfuric acid standard solution consumed, in a unit of mL; 17 is a mass fraction of NH₃, in a unit of g/mol; and V represents a volume of the 2% boric acid-containing absorption solution, in a unit of mL.

Determination method of H₂S: The amount of H₂S released was determined by a zinc-ammonium complex absorption-based colorimetric method. A zinc-ammonium complex was used to absorb the H₂S gas, a mixed chromogenic solution was added, and the resulting mixture was fully shaken and allowed to stand for 30 min. Two drops of a 40% diammonium phosphate solution were added, and the absorbance was measured at a wavelength of 665 nm. A calculation formula was as follows: C=(A−A₀)Bs/Vs. In this formula, C represents a concentration of H₂S, in a unit of mg/m³; A represents the absorbance of a sample chromogenic solution; A₀ represents the absorbance of a blank solution; Bs represents a reciprocal of a slope, in a unit of μg/absorbance; and Vs represents a converted sampling volume under standard conditions, in a unit of L.

Calculation formula for NH₃ and H₂S removal rates: removal rate (%)=(blank concentration−treatment concentration)/blank concentration. An average removal rate of NH₃ and an average removal rate of H₂S were the average removal rate within 8 days.

The reagents used were as follows:

2% boric acid: 10 g of boric acid powder was weighed, and distilled water was added to 500 mL.

0.02 mol/L hydrochloric acid: 1.67 mL of concentrated hydrochloric acid was taken and added to distilled water, and the resulting mixture was thoroughly stirred and diluted to 1,000 mL.

1 mol/L sodium hydroxide solution: 20 g of solid NaOH was dissolved in distilled water and diluted to 500 mL.

0.1% bromocresol green indicator: 1 g of bromocresol green was dissolved in 1,000 mL of 95% ethanol.

0.2% methyl red indicator: 2 g of methyl red was dissolved in 1,000 mL of 95% ethanol.

Methyl red-bromocresol green indicator: The 0.1% bromocresol green indicator and the 0.2% methyl red indicator were mixed in a ratio of 3:1.

Zinc-ammonium complex solution: 5 g of zinc sulfate (ZnSO₄) was dissolved in 500 mL of distilled water to produce a zinc sulfate solution. Six grams of NaOH was dissolved in 300 mL of distilled water to produce a NaOH solution. The zinc sulfate solution and the NaOH solution were mixed. Under stirring, 70 g of ammonium sulfate ((NH₄)₂SO₄) was added, and after dissolution, 50 g of glycerol was added. Distilled water was added to 1 L.

p-aminodimethylaniline stock solution: 50 mL of H₂SO₄ was added to 30 mL of distilled water. When there was no heat release infrom the resulting solution, 12 g of p-aminodimethylaniline hydrochloride was added to the solution.

p-aminodimethylaniline working solution: 2.5 mL of the stock solution was taken and diluted with a 1:1 sulfuric acid solution to 100 mL.

Ferric chloride solution: 100 g of FeCl₃·6H₂O was weighed, dissolved in water, and then diluted to 100 mL.

Mixed chromogenic solution: 1 mL of the p-aminodimethylaniline working solution was mixed with 40 μL of the ferric chloride solution.

40% diammonium phosphate solution: 40 g of (NH₄)₂HPO₄ was weighed, dissolved in distilled water, and then diluted to 100 mL.

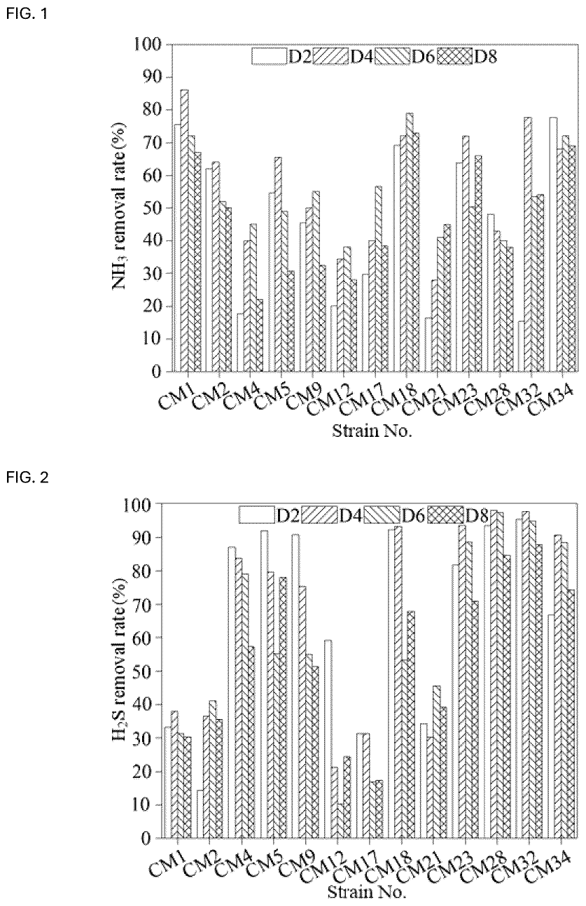

The experiments showed that deodorization effects of different strains varied to some extent, and the strains exhibiting a prominent deodorizing ability in the primary screening also demonstrated the deodorizing ability in the secondary screening, as shown in FIG. 1 FIG. 2, and Table 4.

TABLE 4
Average deodorization rates of different strains
within 8 days of the secondary screening

| | Average removal | Average removal |
|---|---|---|
| Strain No. | rate of NH₃/% | rate of H₂S/% |
| CM1 | 77.79a | 33.12c |
| CM2 | 59.31abc | 31.81c |
| CM4 | 34.19de | 76.77ab |
| CM5 | 56.38bcd | 76.06ab |
| CM9 | 50.15a | 68.08ab |
| CM12 | 30.75e | 28.81c |
| CM17 | 42.10cde | 24.23c |
| CM18 | 73.41a | 76.63ab |
| CM21 | 28.45de | 37.31c |
| CM23 | 62.12ab | 83.59ab |
| CM28 | 43.69cde | 93.26a |
| CM32 | 48.79bcd | 93.82a |
| CM34 | 72.56bcde | 79.92b |

Note:
Different letters in the same column of Table 4 indicate significant differences P < 0.05.

It can be concluded from FIG. 1, FIG. 2, and Table 4 that most strains exhibit a higher removal rate for H₂S than for NH₃. In terms of NH₃ removal, NH₃ removal rates of 7 strains can reach 50% or more, and 2 strains even exhibit the NH₃ removal rate of 70% or higher, indicating a significant inhibitory effect on the release of NH₃. During the entire culture period, the deodorizing abilities of most strains increase initially and then decrease over time. In terms of H₂S removal, the H₂S removal rates for 8 strains can reach 50% or more, and 2 strains even exhibit a H₂S removal rate of 90% or more, indicating an extremely significant inhibitory effect on the release of H₂S.

### 6) Molecular Identification for Deodorizing Strains

A gene fragment was subjected to PCR amplification and sequencing with primer sequences: 27F: 5′-AGAGTTTGATCCTGGCTCAG-3′ (SEQ ID NO: 1) and 1492R: 5′-GGTTACCTTGTTACGACTT-3′ (SEQ ID NO: 2). The 16S rRNA gene sequencing was conducted by Shanghai Majorbio Bio-Pharm Technology Co., Ltd. Sequencing results were compared with sequences in the National Center for Biotechnology Information (NCBI) database for homology analysis, as shown in Table 5. According to the final results, the following three strains were isolated in total: Pseudochrobactrum asaccharolyticum, Providencia sp., and Providencia vermicola, belonging to Pseudochrobactrum asaccharolyticum and Providencia sp.

TABLE 5
Identification of 16 strains at molecule level

| Strain | Similarity | Typical strain with | 16S rRNA gene |
|---|---|---|---|
| No. | degree | the highest homology | sequence |
| CM4 | 99.854% | Pseudochrobactrum | 1369 bp |
| | | asaccharolyticum | |
| CM5 | 100.000% | Pseudochrobactrum | 1361 bp |
| | | asaccharolyticum | |
| CM2 | 99.929% | Providencia sp. | 1407 bp |
| CM9 | 99.930% | Providencia sp. | 1419 bp |
| CM12 | 99.929% | Providencia sp. | 1417 bp |
| CM17 | 100.000% | Providencia sp. | 1413 bp |
| CM18 | 99.929% | Providencia sp. | 1417 bp |
| CM21 | 99.929% | Providencia sp. | 1416 bp |
| CM28 | 99.929% | Providencia sp. | 1412 bp |
| CM32 | 99.929% | Providencia sp. | 1416 bp |
| CM34 | 99.929% | Providencia sp. | 1415 bp |
| CM1 | 100.000% | Providencia vermicola | 1415 bp |
| CM23 | 99.929% | Providencia vermicola | 1410 bp |

### Example 2

### Preparation of an Efficient Composite Deodorizing Microbial Agent

1. Selection of deodorizing strains: By comparing the deodorization rates of different strains in the secondary screening, strains with superior removal abilities for both NH₃ and H₂S were selected.

According to the results of secondary screening in Example 1, among 9 strains of Providencia sp., the CM18 strain exhibited the optimal deodorizing performance, and afforded NH₃ and H₂S removal rates of 70% or higher, which were 73.41% and 76.63%, respectively. CM34 demonstrated a relatively prominent deodorizing effect, and enabled NH₃ and H₂S removal rates of 72.56% and 79.92%, respectively. CM9 also enabled excellent NH₃ and H₂S removal rates of 50.15% and 68.08%, respectively. CM2 demonstrated merely a prominent NH₃ removal effect, with a removal rate of 59.31%. CM28 and CM32 had a H₂S removal rate of 90% or higher, but showed a low NH₃ removal rate of 40%. Other strains, including CM12, CM17, and CM21, all exhibited a general deodorization effect for NH₃ and H₂S, with removal rates of lower than 50%. Therefore, CM18 and CM34 were preferred for the next antagonism test.

2. Antagonism test: The preferred strains were cultured for 24 hours in a liquid medium at 30° C. and 180 r·min⁻¹. 5 μL of strain solution was collected and spotted on a nutrient broth agar medium plate for the antagonism test between every two. The growth of each strain was observed. If two strains could grow normally without affecting each other, there was no antagonism. If there was a distinct growth boundary between two strains, there was antagonism. Several non-antagonistic deodorizing strains were selected. According to deodorization effects of single strains, a strain combination with the optimal NH₃ and H₂S removal effects was determined.

CM18 and CM34 of Providencia sp., CM1 and CM23 of Providencia vermicola, and CM4 and CM5 of Pseudochrobactrum asaccharolyticum were combined pairwise to conduct the antagonism test. Results showed that there was no significant antagonism among the strains. However, CM4 and CM34 grew slowly, and the H₂S removal rate of CM1 was only 33.12%. Ultimately, CM1, CM5, and CM18 were selected for compounding, which were named Providencia vermicola CM1, Pseudochrobactrum asaccharolyticum CM5, and Providencia sp. CM18, respectively.

3. Screening of combinations for the composite deodorizing microbial agent: The selected CM1, CM5, and CM18 were combined pairwise and triply, and an 8-day combination selection experiment was conducted with pig manure. NH₃ and H₂S-absorbing solutions were tested every 2 days to determine the deodorization effect of each strain, and the specific method was the same as the method for the secondary screening of deodorizing strains in Example 1. When two strains were inoculated, these two strains were inoculated at a volume ratio of 1:1, and the total inoculation volume was 10% of the total volume of the medium. When three strains were inoculated, the three strains were inoculated at a volume ratio of 1:1:1, and a total inoculation volume was 10% of a total volume of a medium. Results showed that a combination of the strains CM1, CM5, and CM18 enabled higher average NH₃ and H₂S removal rates than other combinations. Therefore, the combination of CM1, CM5, and CM18 was selected as the final combination for the composite deodorizing microbial agent.

TABLE 6
NH₃ removal rates of different combinations

| | combination | | | |
|---|---|---|---|---|
| | CM1 and | CM1 and | CM5 and | CM1, CM5, |
| Time (day) | CM5 | CM18 | CM18 | and CM18 |
| 2 | 45.22% | 52.07% | 44.77% | 55.00% |
| 4 | 56.74% | 60.46% | 52.74% | 64.67% |
| 6 | 49.44% | 52.81% | 40.33% | 62.33% |
| 8 | 42.57% | 43.59% | 39.26% | 56.84% |
| Average removal | 49.58% | 52.23% | 44.28% | 59.71% |
| rate of NH₃ | | | | |

TABLE 7
H₂S removal rates of different combinations

| | combination | | | |
|---|---|---|---|---|
| | CM1 and | CM1 and | CM5 and | CM1, CM5, |
| Time (day) | CM5 | CM18 | CM18 | and CM18 |
| 2 | 67.33% | 69.67% | 80.44% | 83.00% |
| 4 | 60.91% | 66.75% | 76.25% | 81.27% |
| 6 | 54.29% | 53.25% | 70.57% | 72.77% |
| 8 | 53.78% | 59.85% | 64.38% | 81.09% |
| Average removal | 59.08% | 62.38% | 72.91% | 79.53% |
| rate of H₂S | | | | |

4. Validation of deodorization efficacy and optimization of process parameters for the composite deodorizing microbial agent

The three selected deodorizing strains were each cultured in an NB liquid medium at 30° C. and 180 r·min⁻¹ until a viable count was higher than or equal to 10⁸ cfu/mL. The resulting culture solutions were mixed in equal volumes to produce the composite deodorizing microbial agent, and the composite deodorizing microbial agent was sprayed on pig manure. Different spraying amounts were set to investigate the influence of process parameters on the deodorizing efficacy, thereby determining the spraying plan allowing the optimal deodorization effect.

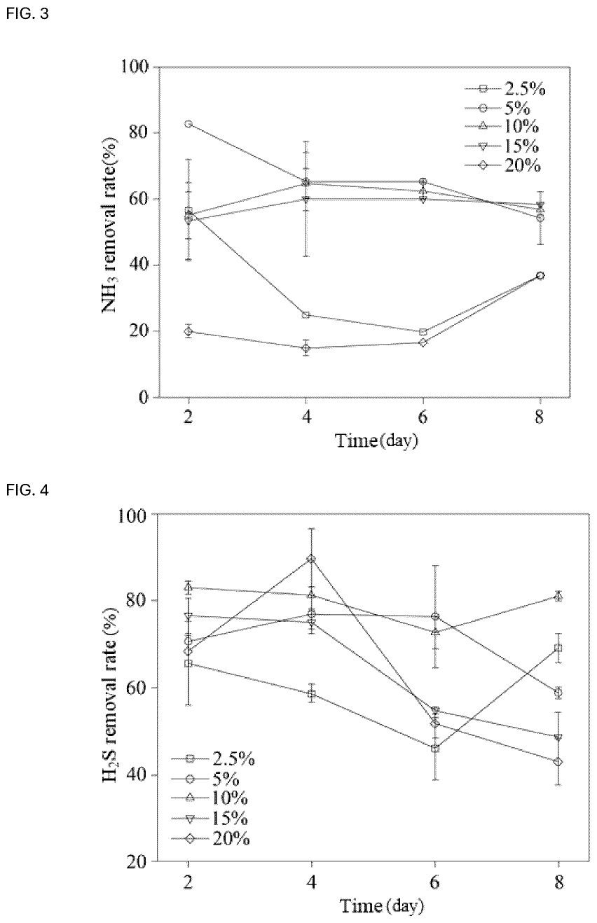

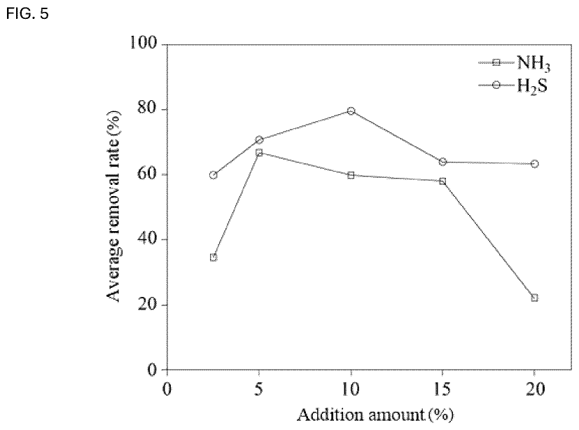

The specific operation method was as follows: 5 spraying amounts were set in the experiment, including 2.5%, 5%, 10%, 15%, and 20% relative to the mass of the pig manure. The entire spraying period was 8 days. NH₃ and H₂S yields at different spraying amounts were measured every 2 days. The amount of NH₃ released was determined by a boric acid absorption-titration method. The amount of H₂S released was determined by a zinc-ammonium complex absorption method. Removal rates of NH₃ and H₂S were subjected to comparative analysis. Three replicates were set for each treatment. Results are shown in FIG. 3 to FIG. 5 and Tables 8 to 10.

TABLE 8
NH₃ removal rates of composite deodorizing microbial
agent at different spraying amounts

| | inoculum size (%) | | | | |
|---|---|---|---|---|---|
| Time (d) | 2.5 | 5 | 10 | 15 | 20 |
| 2 | 56.67% | 82.59% | 55.00% | 53.33% | 20.00% |
| 4 | 25.00% | 65.19% | 64.67% | 60.00% | 15.00% |
| 6 | 20.00% | 65.18% | 62.33% | 60.00% | 16.67% |
| 8 | 36.84% | 54.19% | 56.84% | 58.42% | 36.84% |

TABLE 9
H₂S removal rates of the composite deodorizing
microbial agent at different spraying amounts

| | inoculum size (%) | | | | |
|---|---|---|---|---|---|
| Time (d) | 2.5 | 5 | 10 | 15 | 20 |
| 2 | 65.67% | 70.67% | 83.00% | 77.33% | 68.00% |
| 4 | 58.68% | 76.96% | 81.27% | 75.15% | 89.88% |
| 6 | 46.08% | 76.44% | 72.77% | 54.43% | 50.31% |
| 8 | 69.25% | 58.86% | 81.09% | 48.82% | 40.16% |

TABLE 10
Average removal rates of the composite deodorizing microbial
agent at different spraying amounts within 8 d

| | inoculum size (%) | | | | |
|---|---|---|---|---|---|
| Time (d) | 2.5 | 5 | 10 | 15 | 20 |
| Average removal | 34.63% | 66.79% | 59.71% | 57.94% | 22.13% |
| rate of NH₃ | | | | | |
| Average removal | 59.92% | 70.73% | 79.53% | 63.93% | 62.09% |
| rate of H₂S | | | | | |

According to the removal rates of NH₃ and H₂S in FIG. 3 to FIG. 5 and Tables 8 to 10, the removal rates of NH₃ and H₂S were relatively higher at spraying amounts of 5% and 10% within 8 days, but there was the most stable deodorizing effect at a spraying amount of 10%. The deodorizing ability slowly declined over time. Therefore, when sprayed at an amount of 10%, the composite deodorizing microbial agent exhibited a superior deodorizing effect, with average removal rates of 59.71% for NH₃ and 79.53% for H₂S.

According to the above examples, three novel deodorizing strains were selected in the present disclosure, and these three strains were combined and compounded into a composite deodorizing microbial agent. The composite deodorizing microbial agent showed a high ammonia removal rate and a high sulfur removal rate, and the composite deodorizing microbial agent can be used in the waste treatment at various scenarios such as livestock and poultry farms, household waste composting plants, and landfills, with excellent and stable deodorizing effects.

Although the present disclosure has been described in detail through the above examples, the examples are merely some rather than all of the examples of the present disclosure. All other examples obtained by a person based on these examples without creative efforts shall fall within the protection scope of the present disclosure.

What is claimed is:

1. A deodorizing strain, comprising Providencia vermicola CM1, Pseudochrobactrum asaccharolyticum CM5, or Providencia sp. CM18, wherein accession numbers of the Providencia vermicola CM1, the Pseudochrobactrum asaccharolyticum CM5, and the Providencia sp. CM18 are CGMCC No. 31631, CGMCC No. 31632, and CGMCC No. 31633, respectively.

2. A deodorizing microbial agent comprising a deodorizing strain, wherein
the deodorizing strain comprises one or more strains selected from the group consisting of Providencia vermicola CM1, Pseudochrobactrum asaccharolyticum CM5, and Providencia sp. CM18; and
accession numbers of Providencia vermicola CM1, Pseudochrobactrum asaccharolyticum CM5, and Providencia sp. CM18 are CGMCC No. 31631, CGMCC No. 31632, and CGMCC No. 31633, respectively.

3. The deodorizing microbial agent according to claim 2, wherein the deodorizing microbial agent comprises a microbial suspension of the deodorizing strain and/or a fermentation broth of the deodorizing strain.

4. The deodorizing microbial agent according to claim 3, wherein the deodorizing microbial agent comprises a fermentation broth of Providencia vermicola CM1, a fermentation broth of Pseudochrobactrum asaccharolyticum CM5, and a fermentation broth of Providencia sp. CM18; and the fermentation broth of Providencia vermicola CM1, the fermentation broth of Pseudochrobactrum asaccharolyticum CM5, and the fermentation broth of Providencia sp. CM18 are in a volume ratio of (0.5-2.0):(0.5-2.0):(0.5-2.0).

5. The deodorizing microbial agent according to claim 4, wherein viable counts of Providencia vermicola CM1, Pseudochrobactrum asaccharolyticum CM5, and Providencia sp. CM18 in the deodorizing microbial agent are higher than or equal to 10⁸ cfu/mL.

6. A method for removing odor, comprising spraying the deodorizing microbial agent according to claim 2 on a material carrying the odor, wherein a spraying amount of the deodorizing microbial agent is 5% to 15% of a mass of the material carrying the odor.

7. The method according to claim 6, wherein the material carrying the odor comprises a livestock and poultry manure and/or a household waste.

8. The method according to claim 6, wherein the odor comprises one or more components selected from NH₃, H₂S, and a volatile organic acid

9. The method according to claim 6, wherein the deodorizing microbial agent comprises a microbial suspension of the deodorizing strain and/or a fermentation broth of the deodorizing strain.

10. The method according to claim 6, wherein the deodorizing microbial agent comprises a fermentation broth of Providencia vermicola CM1, a fermentation broth of Pseudochrobactrum asaccharolyticum CM5, and a fermentation broth of Providencia sp. CM18; and the fermentation broth of Providencia vermicola CM1, the fermentation broth of Pseudochrobactrum asaccharolyticum CM5, and the fermentation broth of Providencia sp. CM18 are in a volume ratio of (0.5-2.0):(0.5-2.0):(0.5-2.0).

11. The method according to claim 6, wherein viable counts of Providencia vermicola CM1, Pseudochrobactrum asaccharolyticum CM5, and Providencia sp. CM18 in the deodorizing microbial agent are higher than or equal to 10⁸ cfu/mL.
